(19) United States
(12) Patent Application Publication
(10) Pub. No.: US 2026/0345530 A1
(43) Pub. Date: Nov. 5, 2026

(54) Use of a Therapeutic Agent with Sodium-Hydrogen Antiporter 1 Inhibitory Activity for the Treatment and Prevention of Diseases Associated with Chronic Fatigue, Exhaustion and/or Exertional Intolerance

(71) Applicant: MITODICURE GMBH, Kriftel (DE)

(72) Inventor: Klaus WIRTH, Kriftel (DE)

(21) Appl. No.: 19/106,115

(22) PCT Filed: Aug. 24, 2023
(86) PCT No.: PCT/EP2023/073228

§ 371 Date: Feb. 24, 2025

(30) Foreign Application Priority Data
Aug. 26, 2022 (EP) 22192431.9

(51) Int. Cl. A61K 31/166; A61K 45/06; A61P 21/00

(52) U.S. Cl. CPC A61K 31/166; A61K 45/06; A61P 21/00

(57) ABSTRACT

The instant invention relates to the use of a substance with sodium-hydrogen antiporter 1 inhibitory activity (NHE1 inhibitor) as active ingredient in a therapeutic agent with sodium-hydrogen antiporter 1 inhibitory activity for the treatment and prevention of different diseases, syndromes, disease states, or conditions associated with chronic fatigue, exhaustion and/or exertional intolerance using NHE1 inhibitors.

The instant invention relates to the use of a substance with sodium-hydrogen antiporter 1 inhibitory activity (NHE1 inhibitor) as active ingredient in a therapeutic agent with sodium-hydrogen antiporter 1 inhibitory activity for the treatment and prevention of different diseases, syndromes, disease states, or conditions associated with chronic fatigue, exhaustion and/or exertional intolerance using NHE1 inhibitors.

These diseases, syndromes, disease states, or conditions will subsumed by the term “diseases associated with chronic fatigue” and abbreviated as (DACF) include diseases diagnosed as Myalgic Encephalomyelitis/Chronic Fatigue Syndrome (ME/CFS), systemic exertional intolerance, exertional intolerance, post-vaccination syndrome (post-vac-syndrome) after vaccinations against viruses and pathogenic agents, postinfectious fatigue after viral, bacterial, or fungal infections, cancer-related fatigue (wherein chronic fatigue and exhaustion are symptoms of or are associated with cancer), and fatigue associated with fibromyalgia, Ehlers-Danlos syndrome, Marfan syndrome, Gulf War illness and the autoimmune diseases Rheumatoid Arthritis, ANCA vasculitis (anti-neutrophil cytoplasmic antibodies (ANCA)-associated vasculitis) and Sjögren's syndrome, and other autoimmune diseases with fatigue and exhaustion as debilitating symptoms.

### BACKGROUND OF THE INVENTION

Sodium-proton antiporter 1, also named sodium/proton exchanger 1 or Na⁺/H⁺ exchanger type 1 (abbreviated as NHE1) or SLC9A1 (SoLute Carrier family 9A1), is encoded by the SLC9A1 gene humans (NFliegel et al. doi: 10.1007/BF00936442). The sodium-proton antiporter (SLC9A1) is a ubiquitous membrane-bound transporter involved in volume- and pH-regulation of vertebrate cells. It is activated by a variety of signals including growth factors, mitogens, neurotransmitters, tumor promoters and others (Cardone et al. doi: 10.3390/ijms20153694). In normal conditions, NHE1 maintains intracellular pH (pHi) and volume by removing one intracellular proton (H⁺) ion in exchange for a single extracellular sodium (Na⁺) ion (Fliegel. doi: 10.1016/j.biocel.2004.02.006).

In certain pathological conditions [e.g., heart failure, cardiovascular diseases, diabetes, NHE1 is activated, leading to a rapid accumulation of sodium in cells (Fliegel. doi: 10.3390/ijms20102378) and an acidification of the extracellular space. The high sodium concentration drives the Na⁺/Ca²⁺ exchanger (NCX) as the sodium gradient provides the driving force for the NCX to export calcium (Ca2⁺). In its (physiological) forward mode the NCX exports one calcium ion in exchange for the uptake of three sodium ions controlling cellular calcium. At high sodium concentrations the NCX changes its transport direction (the so-called reverse mode NCX) to import calcium and to export sodium leading to a rise in intracellular calcium and calcium loading. The resulting accumulation of calcium triggers cell damage. NHE1 is strongly responsible for cellular sodium loading that entails deleterious calcium loading. NHE1-inhibitors typically protect from organ damage in ischemia-reperfusion situations which are due to sodium overload that then causes calcium overload by the reverse mode NCX. The involvement of NHE1 in cardiac pathology has been investigated for decades and it is supported by a plethora of experimental studies demonstrating effective NHE1 inhibition not only in protecting the myocardium against ischemic and reperfusion injury but also attenuating myocardial remodeling and heart failure (Evans et al. doi: 10.1016/j.pmrj.2009.04.010). The cardioprotective effects of NHE-1 inhibitors, including Cariporide and Rimeporide, have been extensively studied in various animal models of myocardial infarction and dystrophic cardiomyopathy including DMD (Ghaleh et al. doi: 10.1016/j.ijcard.2020.03.031). Other preclinical experiments (Chahine et al. doi: 10.1016/j.yjmcc.2005.01.003; Bkaily and Jacques, doi: 10.1139/cjpp-2017-0265) have underlined the significance of myocardial necrosis, due to sodium and calcium imbalances in the pathophysiology of heart failure and have demonstrated the beneficial effects of NHE1 inhibition using Cariporide and Rimeporide (CAS 187870-78-6) in preventing the deleterious effects of Ca2⁺ and Na+ overload.

NHE1 inhibition is cardioprotective in experimental models with reduction in cardiac pathology including fibrosis, left ventricular function and improved survival.

NHE1 inhibition leads to normalization of intracellular sodium and calcium, thus improving cellular function; prevention of progressive congestive heart failure; regulation of inflammatory processes; prevention of fibrosis. Known NHE1 inhibitors are for example Cariporide, Rimeporide, Eniporide which all belong to the class of benzoyl-guanidine derivatives (based on a phenyl ring), or amiloride, EIPA (5-(N-ethyl-N-isopropyl)amiloride), DMA (5-(N,N-dimethyl)amiloride), MIBA (5-(N-methyl-N-isobutyl)amiloride) and HMA 5-N, N-(hexamethylene)amiloride which belong to the class of pyrazinoyl-guanidine derivatives (based on a pyrazine ring).

NHE1 is also expressed in skeletal muscle as an important proton-extruder. As in many organs and particularly the heart, NHE1-inhibition has been shown to inhibit ischemia-reperfusion injury in skeletal muscle in animal experiments. This shows its relevance in calcium-overload induced cell damage that is triggered by high intracellular sodium in skeletal muscle. NHE1 strongly participates in cellular sodium loading particularly during exercise to remove the rise in intracellular protons by a 1:1 exchange with sodium ions. Thus, NHE1 enhances intramuscular sodium influx and sodium load. Intracellular sodium is removed and exported from the cell by the Na⁺/K⁺ATPase. If the latter does not work properly intracellular sodium rises with negative consequences for intracellular calcium. The latter can cause detrimental changes in skeletal muscle.

The pathophysiology of diseases associated with chronic fatigue (DACF) had been totally enigmatic so that no rationally based pharmacological or therapeutic strategies could be derived or developed. Therefore, no specific effective treatment exists due to a lack of understanding of these diseases and their pathophysiology. The instant invention has been established by a unifying and comprehensive disease hypothesis elaborated by the instant inventor who found that DACF can be treated by preventing the rise in sodium ions in skeletal muscle. The pathological sodium rise in skeletal muscle has two causes: an increase in sodium influx by NHE1 and a decreased sodium export by the Na⁺/K⁺ATPase. The increased influx is due to the need to export H⁺ ions that are excessively produced in skeletal muscle due to a poor metabolic situation causing anaerobic metabolism. Lactate blood levels are increased in patients with chronic fatigue. Cellular energetic and mitochondrial dysfunction are present in chronic fatigue, particularly in skeletal muscles. Since sodium-proton-exchanger NHE1 imports 1 sodium ion for the export of 1H⁺ intracellular sodium tends to rise when the need for cellular proton export is increased due to a poor metabolic situation (in an anaerobic metabolism). Since the activity of the Na⁺/K⁺ATPase, which removes sodium from the cell cannot cope with the increased sodium influx by the NHE1, sodium rises in the myocytes of the skeletal muscle. The latter entails unfavorable changes in intracellular and mitochondrial calcium (Ca²⁺) in skeletal muscle by changing the activity of the sodium-driven sodium-calcium-exchanger (NCX). These pathological ion changes in skeletal muscle play a strong pathophysiological role in the development and perpetuation of chronic fatigue, exhaustion and/or exertional intolerance. The instant inventor found that the harmful rise in intracellular sodium in skeletal muscle in DACF can be prevented by inhibiting the NHE1 which inhibits sodium influx into skeletal muscle.

In conclusion, inhibition of NHE1 will inhibit sodium overload in myocytes of skeletal muscle thus preventing detrimental changes in calcium that worsen the energetic function of skeletal muscle to cause muscular fatigue and many symptoms of ME/CFS and chronic fatigue.

Among the diseases associated with fatigue, exhaustion, and/or exertional intolerance, Myalgic Encephalomyelitis/Chronic Fatigue Syndrome (ME/CFS) is the most debilitating condition. Although ME/CFS was frequent affecting approximately 0.3% of the population it is not very well-known to most physicians. ME/CFS is characterized by profound fatigue, postexertional malaise (PEM), exertional intolerance, cognitive dysfunction, orthostatic intolerance including postural tachycardia (POTS) and associated with a number of different symptoms including muscle weakness and pain. Despite the fact that non-refreshing sleep is a typical symptom, too, and non-refreshing sleep may worsen the patient's situation, the sleep disturbance is not the initial cause for the profound fatigue and exhaustion, but a consequence of ME/CFS that may further worsen the condition.

Prior to the instant invention, no specific treatment had been available and current symptomatic treatments only provide some symptomatic relief and do not cure. Many patients with ME/CFS are bedridden or wheel chair bound; most are incapacitated for work. ME/CFS is often but not always triggered by infections with various viruses, like Epstein-Barr virus (EBV), enteroviruses, influenza virus, dengue fever.

Hence, there is a strong demand for a drug or therapeutic agent which can be used for the treatment and prevention of chronic fatigue, exhaustion and/or exertional intolerance associated with different diseases, syndromes, disease states (DACF).

Clearly, there is a strong need in the art for new methods for treating and preventing chronic fatigue, exhaustion and/or extertional intolerance associated with different diseases, syndromes, disease states (DACF).

The present invention provides for a new pathway for treating and preventing DACF by the use of a substance sodium-hydrogen antiporter 1 inhibitory activity (NHE1 inhibitor).

As used herein, the term “NHE1 inhibitor” includes chemical compounds, proteins or polypeptides, nucleic acids, ribozymes, DNAzymes, protein degraders, gene therapies, or other such agents, which directly or indirectly inhibit or block the transport activity of the NHE1 protein in a selective or non-selective way. In some cases, the agent may bind or interact directly with NHE1 protein. An agent that binds to NHE1 may act to inhibit or block the NHE1 activity by any suitable means, such as by blocking the ion binding sites. In other cases, the NHE1 inhibitory agent may inhibit NHE1 activity indirectly, such as by decreasing expression of the NHE1 protein. In some cases, the NHE1 inhibitory agent may inhibit NHE1 activity by altering the cellular distribution of NHE1, for example, by interfering with the association between NHE1 and an intracellular anchoring protein.

As used herein, the term diseases associated with chronic fatigue includes different diseases, syndromes, disease states, or conditions associated with chronic fatigue, exhaustion and/or exertional intolerance. It includes Myalgic Encephalomyelitis/Chronic Fatigue Syndrome (ME/CFS), systemic exertional intolerance, exertional intolerance, post-vaccination syndrome (post-vac-syndrome) after vaccinations against viruses and pathogenic agents, postinfectious fatigue and exhaustion after viral, bacterial, or fungal infections. Furthermore, this term includes diseases in which chronic fatigue and exhaustion are symptoms of or associated with cancer (cancer-related fatigue), fibromyalgia, Ehlers-Danlos syndrome, Marfan syndrome, Gulf War illness and the autoimmune diseases Rheumatoid Arthritis, ANCA vasculitis (anti-neutrophil cytoplasmic antibodies (ANCA)-associated vasculitis) and Sjögren's syndrome, and other autoimmune diseases with fatigue and exhaustion as debilitating symptoms. In the following these diseases, syndromes, disease states, syndromes or conditions are subsumed as diseases associated with chronic fatigue (DACF). However, in the instant invention, the term diseases associated with chronic fatigue excludes post-Covid-19 complaints, long Covid and post-Covid-19 syndrome.

In a preferred embodiment of the instant invention the NHE1 inhibitory agent is a compound that is sufficiently potent to inhibit the transport activity of NHE1 at an IC₅₀<50 μM, preferably less than or about 1 μM. Even more preferred, the NHE1 inhibitory agent is sufficiently potent to inhibit the activity of NHE1 at an IC₅₀ of from about 0.1 to about 600 nM, in particular, the NHE1 inhibitory agent is potent to inhibit the enzymatic activity of NHE1 at an IC₅₀ of from about 0.2 to about 100 nM, preferably at an IC₅₀ of from about 1 to about 100 nM.

In the instant invention, the NHE1 inhibitors prevent sodium overload and subsequently calcium overload in skeletal muscle to prevent unfavorable ionic changes that cause mitochondrial and energetic dysfunction in skeletal muscle. The harmful rise in sodium (Na⁺) and ensuing changes in calcium (Ca²⁺) are prevented by NHE1-inhibition. NHE1-inhibition improves the energetic situation in skeletal muscle by preventing the key ionic, metabolic and energetic disturbance in skeletal muscle caused by a rise in sodium (Na⁺) and by preventing subsequent unfavorable changes in cellular and mitochondrial calcium.

The improvements of the energetic situation in skeletal muscle resulting from NHE1-inhibition prevent the release of vasoactive algesic (painful) mediators in skeletal muscles. Normally, these mediators are released to compensate for a poor metabolic situation (raising blood flow to skeletal muscles) and are physiologically meant to act only locally in the skeletal muscle. Since excessively produced in the disease state due to the very poor metabolic situation in the large muscle mass of the body, spillover into the systemic circulation occurs so that these mediators with algesic properties can reach and act on every organ in the body. These mediators cause many of the most different symptoms. Apart from fatigue and exhaustion symptoms associated include brain fog, impaired cognition, disturbed sleep, orthostatic intolerance, muscle weakness, pain, post-exertional malaise (PEM), which is an aggravation of the typical symptoms after small or moderate levels of exercise, and many other complaints.

By these beneficial effects NHE1 inhibition improves the symptoms of DACF and potentially cures the diseases of DACF and prevents the relapses and prevents the diseases in a prophylactic manner.

Specific examples for NHE1 inhibitors used in the instant invention are the hereinafter described substances from the group consisting of the compounds Rimeporide, Cariporide, Eniporide, Amiloride, EIPA (5-(N-ethyl-N-isopropyl)amiloride), DMA (5-(N,N-dimethyl)amiloride), MIBA (5-(N-methyl-N-isobutyl)amiloride), HMA (5-(N, N-(hexamethylene)amiloride), 2-aminophenoxazine-3-one, 2-amino-4,4α-dihydro-4α,7-dimethyl-3H-phenoxazine-3-one, Zoniporide, Compound 9 t, and SL-591227.

Thus, the present invention includes methods of treating or preventing DACF, comprising administering one or more NHE1 inhibitors to a subject having DACF or at risk for developing DACF.

As used herein, unless the context makes clear otherwise, “treat” and similar word such as “treatment” or “treating” etc., is an approach for obtaining beneficial or desired results, including and preferably clinical results. Treatment can involve optionally either the reducing or amelioration of a disease or condition, e.g., DACF, or the delaying of the progression of the disease or condition, e.g., DACF.

As used herein, unless the context makes clear otherwise, “prevent,” and similar word such as “prevention,” “preventing” etc., is an approach for preventing the onset or recurrence of a disease or condition, (e.g., DACF) or preventing the occurrence, e.g., after a viral infection like with the Epstein-Barr virus, or recurrence of the symptoms of a disease or condition, or optionally an approach for delaying the onset or recurrence of a disease or condition or delaying the occurrence or recurrence of the symptoms of a disease or condition.

As used herein, an “effective amount” or a “therapeutically effective amount” of a substance, e.g., a NHE1 inhibitor, is that amount sufficient to affect a desired biological or psychological effect, such as beneficial results, including clinical results. For example, in the context of treating DACF using the methods of the present invention, an effective amount of a NHE1 inhibitor is that amount sufficient to treat and prevent DACF. Generally, a subject is provided with an effective amount of a NHE1 inhibitor.

### Pharmaceutical Compositions, Routes of Administration, Unit Dosage Forms, Kits:

The compositions of the present invention may be administered to a subject as a pharmaceutical composition or formulation. In particular embodiments, pharmaceutical compositions of the present invention may be in any form which allows for the composition to be administered to a subject. For example, the composition may be in the form of a solid, liquid or gas (aerosol). Typical routes of administration include, without limitation, oral, topical, parenteral, sublingual, rectal, vaginal, and intranasal. The term parenteral as used herein includes subcutaneous injections, intravenous, intramuscular, epidural, intrasternal injection or infusion techniques. Pharmaceutical compositions used according to the present invention comprise a NHE1 inhibitor and a pharmaceutically acceptable diluent, excipient, or carrier.

“Pharmaceutically acceptable carriers” for therapeutic use are well known in the pharmaceutical art. For example, sterile saline and phosphate buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

Pharmaceutical compositions of the invention are generally formulated so as to allow the active ingredients contained therein to be bioavailable upon administration of the composition to a subject. Compositions that will be administered to a subject may take the form of one or more dosage units, where for example, a tablet, capsule or cachet may be a single dosage unit, and a container comprising a combination of agents according to the present invention in aerosol form may hold a plurality of dosage units. In particular embodiments, the composition comprising a NHE1 inhibitor and eventually another therapeutic agent is administered in one or more doses of a tablet formulation, typically for oral administration. The tablet formulation may be, e.g., an immediate release formulation, a controlled-release formulation, or an extended-release formulation. In one embodiment, a tablet formulation comprises an effective amount of a composition comprising a NHE1 inhibitor and eventually another therapeutic agent. In particular embodiments, a tablet comprises about 1, 5, 10, 20, 30, 50 100, 150, 200, 250, 300, 400, 500, or 600 mg of a NHE1 inhibitor, and eventually about 1, 5, 10, 20, 30, 50 100, 150, 200, 250, 300, 400, 500, or 600 mg of another therapeutic agent if used in combination.

By way of example, a unit administration form of a compound in accordance with the invention in the form of a tablet can comprise the following components: Rimeporide 300 mg, mannitol 223.75 mg, croscarmellose sodium 6 mg, maize starch 15 mg, hydroxypropylmethylcellulose 2.25 mg, and magnesium stearate 3 mg.

Alternatively, in accordance with the invention a capsule formulation can comprise, by way of example, 300 mg of Rimeporide and pharmaceutically acceptable excipients, wherein said excipients can comprise one or more selected from the group consisting of disintegrators, fillers, and lubricants and can comprise an effective amount of binder.

Preferably, the NHE1 inhibitor is provided to a subject in an amount in the range of 0.1-1000 mg/day, 1-1000 mg/day, 10-100 mg/day, or 25-50 mg/day.

### NHE1 Protein and Inhibitory Agents

In the practice of the methods of the invention, representative NHE1 inhibitory agents that inhibit the activity of NHE1 include: Molecules that bind to NHE1 and inhibit the transporter activity of NHE1 (such as small molecule inhibitors or blocking peptides or proteins/protein fragments/fusion proteins that bind to NHE1 and reduce enzymatic activity), molecules that decrease the expression of NHE1 at the transcriptional and/or translational level (such as NHE1 antisense nucleic acid molecules, NHE1 specific RNAi molecules and NHE1 ribozymes, DNAzymes), and NHE1-directed gene therapies, thereby inhibiting ion transport by NHE1. The NHE1 inhibitory agents can be used alone as a primary therapy or in combination with other therapeutics as an adjuvant therapy to enhance the therapeutic benefits, as discussed here. The inhibition of NHE1 is characterized by at least one of the following changes that occur as a result of administration of a NHE1 inhibitory agent in accordance with the methods of the invention: the inhibition of NHE1-transport activity, a reduction in the gene or protein expression level of NHE1, measured, for example, by gene expression analysis (e.g., RT-PCR analysis) or protein analysis (e.g., Western blot).

In some embodiments, a NHE1 inhibitory agent is a molecule or composition that inhibits the expression of NHE1, such as an antisense or small inhibitory nucleotide (e.g., siRNA) that specifically hybridizes with the cellular mRNA and/or genomic DNA corresponding to the gene(s) of the target NHE1 so as to inhibit its transcription and/or translation, or a ribozyme that specifically cleaves the mRNA of a target NHE1.

### Potency of NHE1 Inhibitory Agents:

In one embodiment, a NHE1 inhibitory agent useful in the methods of the invention is a compound that is sufficiently potent to inhibit the transport activity of NHE1 at an IC₅₀<50 μM, preferably less than or about 1 μM. In one embodiment, the NHE1 inhibitory agent is sufficiently potent to inhibit the activity of NHE1 at an IC₅₀ of from about 0.1 to about 600 nM. In one embodiment, the NHE1 inhibitory agent is potent to inhibit the activity of NHE1 at an IC₅₀ of from about 0.2 to about 100 nM, preferably at an IC₅₀ of from about 1 to about 100 nM. Representative methods for determining the IC₅₀ for a NHE1 inhibitory agent are well known in the art.

### NHE1 Selective Inhibitory Agents:

### Types of NHE1 Inhibitory Agents:

The NHE1 inhibitory agent can be any type of agent including, but not limited to, a chemical compound, a protein or polypeptide, a peptidomimetic, a nucleic acid molecule, a ribozyme, a DNAzyme, a protein degrader, or a gene therapy. In some embodiments, NHE1 inhibitory agents are small molecule inhibitors including natural and synthetic substances that have a low molecular weight, such as, for example, peptides, peptidomimetics and nonpeptide inhibitors such as chemical compounds.

### Chemical Compounds:

The NHE1 inhibitors useful in the methods of the invention include agents that are administered by a conventional route (e.g., oral, intramuscular, subcutaneous, transdermal, transbuccal, intravenous, etc.) into the bloodstream and are ultimately transported through the vascular system to inhibit NHE1 in skeletal muscles and the vasculature.

The following is a description of exemplary NHE1 inhibitors useful in the methods of the invention. They can be orthosteric, allosteric or other inhibitors. If it is not explicitly defined later, the following terms mean:
i) aryl: a functional group derived from an aromatic ring or ring system with or without hetero atoms when one hydrogen is removed from such ring structure,
ii) alkyl: in a broad definition the hydrocarbon group formed when a hydrogen atom is removed from an alkane, alkene, oralkyne group,
iii) alkoxy: functional group containing an alkyl group bonded to oxygen,
iv) acyl: functional group with the formula R—C═O and R being aryl, alkyl or other structures.

NHE1 inhibitors useful in the methods of the invention are selected from known NHE1 inhibitors, for example, Rimeporide, Cariporide, Eniporide which all belong to the class of benzoyl-guanidine derivatives (based on a phenyl ring), or amiloride, EIPA (5-(N-ethyl-N-isopropyl)amiloride), DMA (5-(N,N-dimethyl)amiloride), MIBA (5-(N-methyl-N-isobutyl)amiloride) and HMA 5-N, N-(hexamethylene)amiloride which belong to the class of pyrazinoyl-guanidine derivatives (based on a pyrazine ring) or 2-aminophenoxazine-3-one, 2-amino-4,4α-dihydro-4α,7-dimethyl-3H-phenoxazine-3-one, Zoniporide, Compound 9 t, or SL-591227.

The chemical names and chemical structures of some selected NHE1 inhibitors are as follows:
Rimeporide: N-(4,5-bismethanesulfonyl-2-methylbenzoyl)guanidine (CAS 187870-78-6)

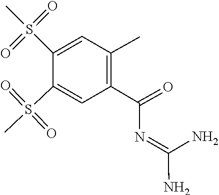 [SMILES: Cc1cc(S(C)(=O)=O)c(S(C)(=O)=O)cc1C(=O)N=C(N)N]

Cariporide: N-Diaminomethylidene)-3-methanesulfonyl-4-(propan-2-yl)benzamide

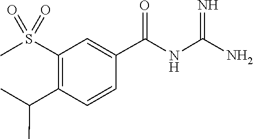 [SMILES: CC(C)c1ccc(C(=O)NC(=N)N)cc1S(C)(=O)=O]

Eniporide: N-(diaminomethylidene)-2-methyl-5-methylsulfonyl-4-pyrrol-1-ylbenzamide

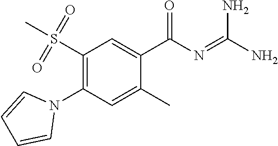 [SMILES: Cc1cc(-n2cccc2)c(S(C)(=O)=O)cc1C(=O)N=C(N)N]

Amiloride: 3,5-Diamino-N-carbamimidoyl-6-chlorpyrazin-2-carbamide

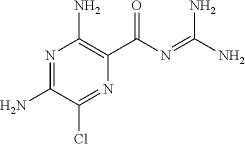 [SMILES: NC(N)=NC(=O)c1nc(Cl)c(N)nc1N]

HMA (5-(N, N-hexamethylene) amiloride)

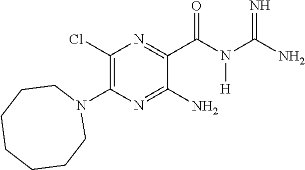 [SMILES: N=C(N)NC(=O)c1nc(Cl)c(N2CCCCCCC2)nc1N]

EIPA (5-(N-ethyl-N-isopropyl)amiloride)

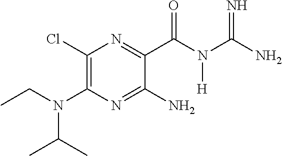 [SMILES: CCN(c1nc(N)c(C(=O)NC(=N)N)nc1Cl)C(C)C]

DMA (5-(N,N-dimethyl)amiloride)

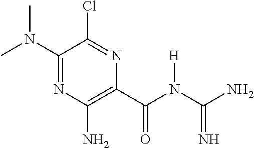 [SMILES: CN(C)c1nc(N)c(C(=O)NC(=N)N)nc1Cl]

MIBA (5-(N-methyl-N-isobutyl)amiloride)

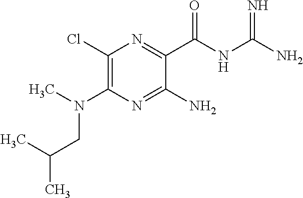 [SMILES: CC(C)CN(C)c1nc(N)c(C(=O)NC(=N)N)nc1Cl]

An aminophenoxazine derivative: 2-Aminophenoxazine-3-one

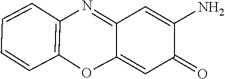 [SMILES: Nc1cc2nc3ccccc3oc-2cc1=O]

Another aminophenoxazine derivative: 2-Amino-4,4α-dihydro-4α,7-dimethyl-3H-phenoxazine-3-one

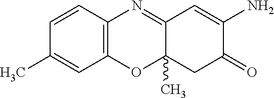 [SMILES: Cc1ccc2c(c1)OC1(C)CC(=O)C(N)=CC1=N2]

Zoniporide

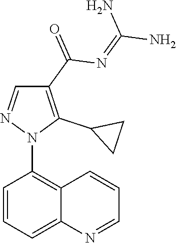 [SMILES: NC(N)=NC(=O)c1cnn(-c2cccc3ncccc23)c1C1CC1]

Compound 9 t (CAS 335063-97-3)
5-(4-Fluoro-3-methylphenyl)-2-methoxy-4-[4-(5-methyl-1H-imidazol-4-yl)piperidin-1-yl]pyrimidine

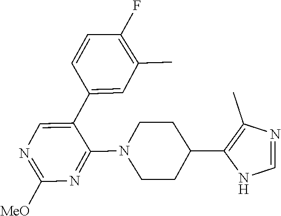 [SMILES: COc1ncc(-c2ccc(F)c(C)c2)c(N2CCC(c3[nH]cnc3C)CC2)n1]

SL-591227 (CAS 326488-60-2)
3-[(Cyclopropylcarbonyl)amino]-N-[2-(dimethylamino)ethyl]-4-[4-(4-methyl-1H-imidazol-5-yl)-1-piperidinyl]benzamide

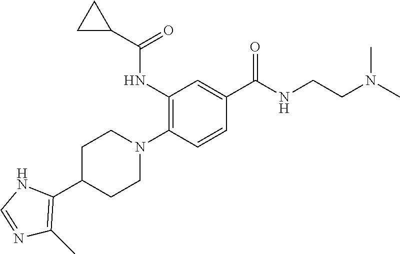 [SMILES: Cc1nc[nH]c1C1CCN(c2ccc(C(=O)NCCN(C)C)cc2NC(=O)C2CC2)CC1]

Or as a pharmaceutically accepted salt like DMA (5-(N,N-dimethyl)amiloride) (hydrochloride)

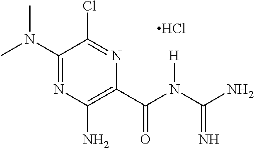 [SMILES: CN(C)c1nc(N)c(C(=O)NC(=N)N)nc1Cl ; Cl]

or Zoniporide (hydrochloride)

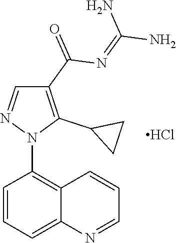 [SMILES: NC(N)=NC(=O)c1cnn(-c2cccc3ncccc23)c1C1CC1 ; Cl]

In another embodiment, NHE1 inhibitors useful in the methods of the invention are selected from the pyrrolidinyl and piperidinyl compounds generally or specifically disclosed in WO 2010/005783, expressly incorporated herein by reference in its entirety.

### Proteins, Polypeptides or Peptide Inhibitors:

In some embodiments, the NHE1 inhibitory agent comprises isolated NHE1 polypeptide or peptide inhibitors, including isolated natural peptide inhibitors and synthetic peptide inhibitors that inhibit NHE1 activity. As used herein, the term “isolated NHE1 polypeptide or peptide inhibitors” refers to polypeptides or peptides that inhibit NHE1, competing with NHE1 for binding to ions, and/or directly interacting with NHE1 to inhibit NHE1-transport activity, that are substantially pure and are essentially free of other substances with which they may be found in nature to an extent practical and appropriate for their intended use.

Peptide inhibitors have been used successfully in vivo to interfere with protein-protein interactions and binding sites. For example, peptide inhibitors to adhesion molecules structurally related to LFA-1 have been approved for clinical use in coagulopathies. Short linear peptides (<30 amino acids) have been described that prevent or interfere with integrin-dependent adhesion. Longer peptides, ranging in length from 25 to 200 amino acid residues, have also been used successfully to block integrin-dependent adhesion. In general, longer peptide inhibitors have higher affinities and/or slower off-rates than short peptides and may therefore be more potent inhibitors. Cyclic peptide inhibitors have also been shown to be effective inhibitors of integrins in vivo for the treatment of human inflammatory disease. One method of producing cyclic peptides involves the synthesis of peptides in which the terminal amino acids of the peptide are cysteines, thereby allowing the peptide to exist in a cyclic form by disulfide bonding between the terminal amino acids, which has been shown to improve affinity and half-life in vivo for the treatment of hematopoietic neoplasms.

NHE1-binding proteins, antibodies, nanobodies or functionally related proteins or protein fragments or fusion proteins like single-chain variable fragments or DARPins (Designed Ankyrin Repeat Proteins) or lipocalins/anticalins or other such molecules, that enter cells or are modified/conjugated with other moieties so that they can enter cells, also inhibit NHE1.

### Synthetic NHE1 Peptide Inhibitors

NHE1 inhibitory peptides useful in the methods of the invention are exemplified by amino acid sequences that mimic the target regions important for NHE1 transporter activity, such as the sodium binding domain of NHE1. One may also use molecular modeling and rational molecular design to generate and screen for peptides that mimic the molecular structure of the NHE1 binding regions and inhibit the transporter activity of NHE1. The molecular structures used for modeling include the CDR regions of anti-NHE1 monoclonal antibodies. Methods for identifying peptides that bind to a particular target are well known in the art. For example, molecular imprinting may be used for the de novo construction of macromolecular structures such as peptides that bind to a particular molecule. See, for example, Shea, K. J., “Molecular Imprinting of Synthetic Network Polymers: The De Novo Synthesis of Macromolecular Binding and Catalytic Sites,” Trends in Polymer Science 2(5):166-173 (1994).

As an illustrative example, one method of preparing mimics of NHE1 binding peptides is as follows. Functional monomers of a binding region of an anti-NHE1 antibody that exhibits NHE1 inhibition (the template) are polymerized. The template is then removed, followed by polymerization of a second class of monomers in the void left by the template, to provide a new molecule that exhibits one or more desired properties that are similar to the template. In addition to preparing peptides in this manner, other NHE1 binding molecules that are NHE1 inhibitory agents, such as polysaccharides, nucleosides, drugs, nucleoproteins, lipoproteins, carbohydrates, glycoproteins, steroids, lipids, and other biologically active materials, can also be prepared. This method is useful for designing a wide variety of biological mimics that are more stable than their natural counterparts because they are typically prepared by free radical polymerization of functional monomers, resulting in a compound with a nonbiodegradable backbone.

The NHE1 inhibitory peptides can be prepared using techniques well known in the art, such as the solid-phase synthetic technique initially described by Merrifield in J. Amer. Chem. Soc. 85:2149-2154, 1963. Automated synthesis may be achieved, for example, using Applied Biosystems 431A Peptide Synthesizer (Foster City, Calif.) in accordance with the instructions provided by the manufacturer. Other techniques may be found, for example, in Bodanszky, M., et al., Peptide Synthesis, second edition, John Wiley & Sons, 1976, as well as in other reference works known to those skilled in the art. The peptides can also be prepared using standard genetic engineering techniques known to those skilled in the art.

A candidate NHE1 inhibitory peptide may be tested for the ability to function as a NHE1 inhibitory agent in one of several assays, including, for example, a NHE1 cellular assay.

### Expression Inhibitors of NHE1, Nucleic Acids

In some embodiments of the methods of the invention, the NHE1 inhibitory agent is a NHE1 expression inhibitor capable of inhibiting NHE1-dependent sodium and hydrogen transport. In the practice of this embodiment of the invention, representative NHE1 expression inhibitors include NHE1 antisense nucleic acid molecules (such as antisense mRNA, antisense RNA, antisense DNA, or antisense oligonucleotides), NHE1 RNAi molecules, NHE1 ribozymes, and NHE1 DNAzymes.

Anti-sense RNA and DNA molecules act to directly block the translation of NHE1 mRNA by hybridizing to NHE1 mRNA and preventing translation of NHE1 protein. An antisense nucleic acid molecule may be constructed in a number of different ways provided that it is capable of interfering with the expression of NHE1. For example, an antisense nucleic acid molecule can be constructed by inverting the coding region (or a portion thereof) of NHE1 cDNA relative to its normal orientation for transcription to allow for the transcription of its complement. Methods for designing and administering antisense oligonucleotides are well known in the art and are described, e.g., in Mautino et al., Hum Gene Ther 13:1027-37, 2002; and Pachori et al., Hypertension 39:969-75, 2002, each of which is hereby incorporated by reference.

The antisense nucleic acid molecule is usually substantially identical to at least a portion of the target gene or genes. The nucleic acid, however, need not be perfectly identical to inhibit expression. Generally, higher homology can be used to compensate for the use of a shorter antisense nucleic acid molecule. The minimal percent identity is typically greater than about 65%, but a higher percent identity may exert a more effective repression of expression of the endogenous sequence. Substantially greater percent identity of more than about 80% typically is preferred, though about 95% to absolute identity is typically most preferred.

The antisense nucleic acid molecule need not have the same intron or exon pattern as the target gene, and non-coding segments of the target gene may be equally effective in achieving antisense suppression of target gene expression as coding segments. A DNA sequence of at least about 8 or so nucleotides may be used as the antisense nucleic acid molecule, although a longer sequence is preferable. In the present invention, a representative example of a useful inhibitory agent of NHE1 is an antisense NHE1 nucleic acid molecule that is at least ninety percent identical to the complement of a portion of the NHE1 cDNA.

The targeting of antisense oligonucleotides to bind NHE1 mRNA is another mechanism that may be used to reduce the level of NHE1 protein synthesis. For example, U.S. Pat. No. 7,579,455 to Paolo or the synthesis of polygalacturonase and the muscarine type 2 acetylcholine receptor is inhibited by antisense oligonucleotides directed to their respective mRNA sequences (U.S. Pat. No. 5,739,119 to Galli, and U.S. Pat. No. 5,759,829 to Shewmaker). Furthermore, examples of antisense inhibition have been demonstrated with the nuclear protein cyclin, the multiple drug resistance gene (MDG1), ICAM-1, E-selectin, STK-1, striatal GABA, receptor and human EGF (see, e.g., U.S. Pat. No. 5,801,154 to Baracchini; U.S. Pat. No. 5,789,573 to Baker; U.S. Pat. No. 5,718,709 to Considine; and U.S. Pat. No. 5,610,288 to Rubenstein).

A system has been described that allows one of ordinary skill to determine which oligonucleotides are useful in the invention, which involves probing for suitable sites in the target mRNA using Rnase H cleavage as an indicator for accessibility of sequences within the transcripts. Scherr, M., et al., Nucleic Acids Res. 26:5079-5085, 1998; Lloyd, et al., Nucleic Acids Res. 29:3665-3673, 2001. A mixture of antisense oligonucleotides that are complementary to certain regions of the NHE1 transcript is added to cell extracts expressing NHE1 and hybridized in order to create an RNAseH vulnerable site. This method can be combined with computer-assisted sequence selection that can predict optimal sequence selection for antisense compositions based upon their relative ability to form dimers, hairpins, or other secondary structures that would reduce or prohibit specific binding to the target mRNA in a host cell. These secondary structure analysis and target site selection considerations may be performed using the OLIGO primer analysis software (Rychlik, I., 1997) and the BLASTN 2.0.5 algorithm software (Altschul, S. F., et al., Nucl. Acids Res. 25:3389-3402, 1997). The antisense compounds directed towards the target sequence preferably comprise from about 8 to about 50 nucleotides in length. Antisense oligonucleotides comprising from about 9 to about 35 or so nucleotides are particularly preferred. The inventor contemplates all oligonucleotide compositions in the range of 9 to 35 nucleotides (i.e., those of 9, 10, 11, 12, 13, 14, 15, 16, 17, 18, 19, 20, 21, 22, 23, 24, 25, 26, 27, 28, 29, 30, 31, 32, 33, 34, or 35 or so bases in length) are highly preferred for the practice of antisense oligonucleotide-based methods of the invention. Highly preferred target regions of the NHE1 mRNA are those that are at or near the AUG translation initiation codon, and those sequences that are substantially complementary to 5′ regions of the mRNA, e.g., between the 0 and +10 regions of the NHE1 gene nucleotide sequence.

The term “oligonucleotide” as used herein refers to an oligomer or polymer of ribonucleic acid (RNA) or deoxyribonucleic acid (DNA) or mimetics thereof. This term also covers those oligonucleobases composed of naturally occurring nucleotides, sugars and covalent internucleoside (backbone) linkages as well as oligonucleotides having non-naturally occurring modifications. These modifications allow one to introduce certain desirable properties that are not offered through naturally occurring oligonucleotides, such as reduced toxic properties, increased stability against nuclease degradation and enhanced cellular uptake.

Another alternative to antisense is the use of “RNA interference” (RNAi). Double-stranded RNAs (dsRNAs) can provoke gene silencing in mammals in vivo. The natural function of RNAi and co-linsuppression appears to be protection of the genome against invasion by mobile genetic elements such as retrotransposons and viruses that produce aberrant RNA or dsRNA in the host cell when they become active (see, e.g., Jensen, J., et al., Nat. Genet. 21:209-12, 1999). The double-stranded RNA molecule may be prepared by synthesizing two RNA strands capable of forming a double-stranded RNA molecule, each having a length from about 19 to 25 (e.g., 19-23 nucleotides). For example, a dsRNA molecule useful in the methods of the invention may comprise the RNA corresponding to a portion of at least one of human NHE1 and its complement. Preferably, at least one strand of RNA has a 3′ overhang from 1-5 nucleotides. The synthesized RNA strands are combined under conditions that form a double-stranded molecule. The RNA sequence may comprise at least an 8 nucleotide portion of human NHE1 with a total length of 25 nucleotides or less. The design of siRNA sequences for a given target is within the ordinary skill of one in the art. Commercial services are available that design siRNA sequence and guarantee at least 70% knockdown of expression (Qiagen, Valencia, Calif.). Exemplary NHE1 shRNAs and siRNAs are commercially available from Sigma-Aldrich Company (product #SHDNA_-NM_002603; SASI_Hs01_00183420 to SASI_Hs01_00010490).

The dsRNA may be administered as a pharmaceutical composition and carried out by known methods, wherein a nucleic acid is introduced into a desired target cell. Commonly used gene transfer methods include calcium phosphate, DEAE-dextran, electroporation, microinjection and viral methods. Such methods are taught in Ausubel et al., Current Protocols in Molecular Biology, John Wiley & Sons, Inc., 1993.

### Ribozymes and DNAzymes

In some embodiments, a NHE1 inhibitory agent is a ribozyme or a DNAzyme that specifically cleaves the mRNA of a target NHE1 or a NHE1 itself. Ribozymes that target NHE1 may be utilized as NHE1 inhibitory agents to decrease the amount and/or biological activity of NHE1. Ribozymes are catalytic RNA molecules that can cleave nucleic acid molecules having a sequence that is completely or partially homologous to the sequence of the ribozyme. It is possible to design ribozyme transgenes that encode RNA ribozymes that specifically pair with a target RNA and cleave the metester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs.

Ribozymes useful in the practice of the invention typically comprise a hybridizing region of at least about nine nucleotides, which is complementary in nucleotide sequence to at least part of the target NHE1 mRNA, and a catalytic region that is adapted to cleave the target NHE1 mRNA (see generally, European Patent No. 0 321 201; WO 88/04300; Haseloff, J., et al., Nature 334:585-591, 1988; Fedor, M. J., et al., Proc. Natl. Acad. Sci. USA 87:1668-1672, 1990; Cech, T. R., et al., Ann. Rev. Biochem. 55:599-629, 1986).

Ribozymes can either be targeted directly to cells in the form of RNA oligonucleotides incorporating ribozyme sequences, or introduced into the cell as an expression vector encoding the desired ribozymal RNA. Ribozymes may be used and applied in much the same way as described for antisense polynucleotides.

Anti-sense RNA and DNA, RNAi molecules, ribozymes, and DNAzymes useful in the methods of the invention may be prepared by any method known in the art for the synthesis of DNA and RNA molecules. These include techniques for chemically synthesizing oligodeoxyribonucleotides and oligoribonucleotides well known in the art, such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications of the DNA molecules may be introduced as a means of increasing stability and half-life. Useful modifications include, but are not limited to, the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

### Protein Degraders

Protein degradation technologies also decrease or inhibit NHE1 activity. Many technologies are in use like proteolysis targeting chimeras (PROTACs), which are bispecific molecules containing a (here NHE1) target protein binder and an ubiquitin ligase binder connected by a linker or other degradation technologies comprising PROTACs, SNIPERs, HaloPROTACs, HyTs, LYTACs, AUTACs, ATTECs, RIBOTECs, monomeric degraders, double-mechanism degraders, SARDs, TF-PROTACs, dual-PROTACs, SERDs, bispecific aptamer chimeras, AbTACs, GlueTACs, AUTOTACs, CMA-based degraders, MADTACs, ATACs, molecular glues, biodegraders, mRNAs for the biodegrader protein, mRNAs for the biodegrader protein in a lipid nanoparticle formulation, all directed at NHE1 (for example Li, H. et al., J Hematol Oncol 14(1):138, 2021).

### Gene Therapies

### Modification, Complementation, Replacement, or Deletion of the NHE1 Genes in Selected Tissues Also Result in Inhibiting the Respective NHE1 Activity.

The invention provides a method of treating diseases associated with chronic fatigue (DACF) by administering it to a patient in need thereof an amount of a NHE1 inhibitory agent effective to inhibit the transport activity of NHE1, wherein such inhibition of NHE1 activity is the principal therapeutic mode of action of the NHE1 inhibitor in the treatment of DACF.

For each of the NHE1 inhibitory chemical compounds useful in the method of the present invention, all possible stereoisomers and geometric isomers are included. The compounds include not only racemic compounds, but also the optically active isomers. When a NHE1 inhibitory agent is desired as a single enantiomer, it can be obtained either by resolution of the final product or by stereospecific synthesis from either isomerically pure starting material or use of a chiral auxiliary reagent, for example, see Ma, Z., et al., Tetrahedron: Asymmetry 8(6):883-888, 1997. Resolution of the final product, an intermediate, or a starting material can be achieved by any suitable method known in the art. Additionally, in situations where tautomers of the compounds are possible, the present invention is intended to include all tautomeric forms of the compounds.

The NHE1 inhibitory agents that contain acidic moieties can form pharmaceutically acceptable salts with suitable cations. Suitable pharmaceutically acceptable cations include alkali metal (e.g., sodium or potassium) and alkaline earth metal (e.g., calcium or magnesium) cations. The pharmaceutically acceptable salts of the NHE1 inhibitory agents, which contain a basic center, are acid addition salts formed with pharmaceutically acceptable acids. Examples include the hydrochloride, hydro bromide, sulfate or bisulfate, phosphate or hydrogen phosphate, acetate, benzoate, succinate, fumarate, maleate, lactate, citrate, tartarate, gluconate, methanefulgonate, bezenesulphonate, and p-toluenesulphonate salts. In light of the foregoing, any reference to compounds useful in the method of the invention appearing herein is intended to include NHE1 inhibitory agents, as well as pharmaceutically acceptable salts and solvates thereof.

The compounds of the present invention can be therapeutically administered as the neat chemical, but it is preferable to administer the NHE1 inhibitory agents as a pharmaceutical composition or formulation. Accordingly, the present invention further provides for pharmaceutical compositions or formulations comprising a NHE1 inhibitory agent, or pharmaceutically acceptable salts thereof, together with one or more pharmaceutically acceptable carriers and, optionally, other therapeutic and/or prophylactic ingredients. Suitable carriers are compatible with the other ingredients of the formulation and not deleterious to the recipient thereof. Compounds of the present invention may also be carried in a delivery system to provide for sustained release or enhanced uptake or activity of the compound, such as a liposomal or hydrogel system for injection, a microparticle, nanopartical, or micelle system for oral or parenteral delivery, or a staged capsule system for oral delivery.

### EXAMPLE

The following example describes the use of some compounds in accordance with the invention. These examples are not limiting and serve only to illustrate the present invention.

### Example 1—Ex Vivo Model on More Residual Muscle Force (Less Fatigue) Via Inhibition of Sodium Overload and Detrimental Cellular Calcium Changes and Calcium Overload in Skeletal Muscles

### Introduction of the Model

The claim of this patent is that NHE1 inhibitors can improve disturbed body function and symptoms of ME/CFS, as an example of diseases associated with chronic fatigue (DACF). Muscular fatigue and loss of force (e.g., loss of handgrip strength) is a hallmark in ME/CFS patients. A main target of NHE1 inhibition is the skeletal muscle whose energetic function is deeply disturbed in ME/CFS and which is presumed to be very important in the symptomatology of ME/CFS. NHE1 activity plays a key role in the development of the disturbances by a rise in intramuscular sodium that finally leads to calcium overload. The latter causes a deep functional energetic disturbance or damage. The isolated rat muscle either soleus muscle or extensor digitorum longus muscle is a well-characterized and well-validated model for the study of the function of ion exchangers. The isolated rat soleus muscle is particularly appropriate for experiments ex vivo as it is very thin so that there is no need for blood supply. It has been demonstrated under different experimental conditions that cellular sodium ion export reduces the decline in contractile force that occurs after measures such as carbachol application or high ion concentrations, e.g., 12.5 mM KCl, or by worsening of the energetic situation during electrical stimulation or by a high extracellular NaCl concentration that results in cellular sodium overload. In this experimental setting, a high NaCl concentration buffer causes sodium influx to disturb and decrease excitability and to disturb calcium homeostasis to subsequently reduce muscle force. Inhibition of the NHE1, which exchanges one proton for the uptake of one sodium ion, reduces sodium entry and overload. Prevention or diminution of a loss of muscular force in this procedure indicates effective inhibition of NHE1 by a test compound with the described mode of action of NHE1-inhibition.

The decline in force in the soleus muscle can be considered as muscular fatigue and related to the loss of hand grip muscular strength in ME/CFS patients. The described ex vivo model is suited for testing the effect of NHE1 inhibitors and in particular their effect on force generation disturbed by mechanisms that worsen excitability or the energetic situation or calcium homeostasis via the sodium-calcium-exchanger. High NaCl in a buffer (200 mM) is used to cause intracellular sodium loading leading to a decline in contractile force. Electrical stimulations performed at physiological extracellular NaCl concentration after a period of exposure to high NaCl buffer, shows a decline in force.

The resulting decline in muscular force is considered as muscular fatigue. In the absence of electrical stimulation avoiding sodium entry by sodium channels the sodium influx during high NaCl exposure is due to the NHE1. Inhibition of NHE1 can prevent sodium overload and a decline in muscular force and fatigue.

### Method

Muscle preparation and incubation: Female Sprague Dawley rats of 5 weeks of age are used. After isoflurane anesthesia and cervical dislocation the intact soleus muscles of the animals is dissected. The soleus muscles is mounted at resting length in a vertical position between two field stimulation electrodes (thick copper wires) in organ bathes containing 15 mL incubation medium. The lower end of the muscle is fixed to a clip fixed to the bottom of the organ bath, the upper end of the muscle is fixed to an isometric force transducer (Force transducer K300 Hugo Sachs) by a clip allowing measurement of isometric contractions, and a tension of 100 mN is applied.

Force measurement: The experimental set-up for force measurements allows for the simultaneous recordings from 6 muscles in separate incubation chambers in parallel. Force is recorded and stored with a Notocord HEM data acquisition system. The standard incubation medium is a Krebs-Ringer (KR) bicarbonate buffer (pH 7.4) containing (in mM): 120.1 NaCl, 25.1 NaHCO₃, 4.7 KCl, 1.2 KH₂PO₄, 1.2 MgSO₄, 1.3 CaCl₂ and 5 glucose. Incubation takes place at 30 °C. The buffer is continuously gassed with a mixture of 95% O₂ and 5% CO₂. Muscles are equilibrated with a mixture of 95% O₂ and 5% CO₂ in the standard medium for 30 minutes (min) before stimulation.

Electrical stimulation protocol: After an equilibration period of 30 min muscles are field-stimulated to deliver optimal force at 83 Hz, 2-3 ms pulses, 12 V (biphasic), 0.2-0.5 s train width with a Hugo Sachs stimulator (HSE). After optimizing for the stimulation protocol, the muscles are stimulated every 10 min for 30 min in the buffer indicated above to obtain the control values of force before weakening the contractile force by exposure to high NaCl in the buffer.

Investigation of test compounds: At time zero (TO) the buffer described above is exchanged against a buffer containing NaCl 200 mM. No electrical stimulation is performed for the next 30 min. Then the buffer is exchanged for the initial buffer with the physiological NaCl concentration and electrical stimulation is performed 1 min later. The high NaCl buffer causes a strong loss of force in controls.

NHE1 inhibitors are added in a volume of 0.1 mL to the organ baths containing 15 mL buffer solution already 10 min before exchanging (10 min before TO) the initial buffer for the new buffer with high NaCl of 200 mM and they are newly added to the buffer with physiological NaCl after the exchange. Then electrical stimulation is performed. Electrical stimulations are only performed in the presence of physiological NaCl concentrations; buffer with high NaCl only serves to load the cells with sodium. A control group receives buffer only instead of the NHE1 inhibitors.

### Evaluation

High NaCl 200 mM causes a strong loss of force in controls. An effective drug would show a lower loss of contractile force, thus preserve contractile force. The residual contractile force after 30 min in high NaCl buffer is chosen as the efficacy parameter and related to the last value of stimulated force in the initial buffer in the same organ bath. The percentages of residual force are compared between the different groups and used for statistical calculation.

### Results

As illustrative and nonlimiting examples, NHE1 inhibitor compounds in accordance with the invention were tested according to the above protocol and delays the rat soleus muscle force decline, thus, showing the advantage as therapeutically active substance.

Soleus muscle force strongly declined under the effect of a high extracellular sodium concentration (200 mM) to 10% to 20% of the force measured at normal sodium ion concentration at baseline. The NHE1 inhibitors Cariporide and Rimeporide at drug concentrations of 30 to 500 nM diminished the loss of force and, thus, showed preservation of muscular force with residual forces of 50-70% compared with their baseline controls.

### CONCLUSION

NHE1 inhibitors reduce muscular fatigue by a preservation of contractile force under conditions of sodium overload caused by a high NaCl buffer. The mechanism is via reducing sodium import and overload by NHE1 inhibition, while still some sodium export by the Na⁺/K⁺ATPase takes place. Based on these actions NHE1 inhibitors can treat muscular fatigue and loss of force. Moreover, due to NHE1 inhibition lowering intracellular sodium—high intramuscular sodium causes calcium overload via the reverse mode of the sodium-calcium-exchanger (NCX) to cause muscular damage and mitochondrial dysfunction—NHE1 inhibitors can treat and prevent mitochondrial dysfunction and the deleterious secondary consequences of energy deprivation. This deprivation otherwise results in the physiological stimulation of the production of vasoactive mediators from skeletal muscle, which are normally physiologically meant to raise muscular blood flow. These mediators are then excessively produced due to the very poor energetic situation and therefore released into the systemic blood stream (spillover). Thus, these mediators with algesic, hyperalgesic, spasmogenic and microvascular leakage-inducing properties can reach every organ to cause a variety of symptoms of diseases associated with chronic fatigue. These include pain, edema and spasms, which are prevented by treatment involving NHE1 inhibition in the form of an indirect effect.

1. Substance with NHE1 inhibitory activity (NHE1-inhibitor) for use as active ingredient in a therapeutic agent with sodium-hydrogen antiporter 1 inhibitory activity for the treatment and prevention of chronic fatigue, exhaustion and/or exertional intolerance and for the treatment and prevention of diseases that are associated with chronic fatigue, exhaustion and/or exertional intolerance.

2. Substance as claimed in claim 1, wherein diseases that are associated with chronic fatigue, exhaustion and/or exertional intolerance are diseases diagnosed as Myalgic Encephalomyelitis/Chronic Fatigue Syndrome (ME/CFS), systemic exertional intolerance, exertional intolerance, post-vaccination syndrome (post-vac-syndrome) after vaccinations against viruses and pathogenic agents, postinfectious fatigue after viral, bacterial, or fungal infections excluding post-Covid-19 complaints, long Covid and post-Covid-19 syndrome.

3. Substance as claimed in claim 1, wherein chronic fatigue and exhaustion are symptoms of or are associated with cancer (cancer-related fatigue), fibromyalgia, Ehlers-Danlos syndrome, Marfan syndrome, Gulf War illness, the autoimmune diseases Rheumatoid Arthritis, ANCA vasculitis and Sjögren's syndrome, and other autoimmune diseases with fatigue and exhaustion as debilitating symptoms.

4. The substance as claimed in one or more of claims 1 to 3, wherein the NHE1 inhibitory material is a material that inhibits the transport activity of NHE1 at an IC₅₀<50 μM, preferably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.

5. The substance as claimed in one or more of claims 1 to 4, wherein the NHE1 inhibitory agent inhibits sodium overload and detrimental cellular calcium changes and calcium overload in skeletal muscles.

6. The substance as claimed in one or more of claims 1 to 5, wherein the NHE1 inhibitory agent is present in a pharmaceutical preparation, preferably in the form of an oral drug, intravenous, subcutaneous, intramuscular, pharyngeal, and nasal administration.

7. The substance as claimed in one or more of claims 1 to 6, wherein the NHE1 inhibitory agent is a chemical compound, a protein or polypeptide, a nucleic acid molecule, ribozyme, DNAzyme, a protein degrader, or a material for gene therapy.

8. The substance as claimed in claim 7, wherein the chemical compound is a small molecule inhibitor including natural and synthetic substances that have a low molecular weight, such as, a peptide, a peptidomimetic and a non-peptide inhibitor such as a classical small molecule chemical compound, preferably the small molecule inhibitor having a molecular weight of less than about 750 g/mol and an IC₅₀ for inhibiting NHE1 activity at an IC₅₀<50 μM, preferably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.

9. The substance as claimed in claim 7, wherein the NHE1 inhibitory agent is a protein or polypeptide including a NHE1-binding antibody, nanobody, or functionally related protein or protein fragment or fusion protein, including a single-chain variable fragment or a designed ankyrin repeat protein or lipocalin/anticalin.

10. The substance as claimed in claim 7, wherein the NHE1 inhibitory agent inhibits NHE1 expression and is an antisense nucleic acid molecule such as antisense oligonucleotide, antisense RNA, antisense mRNA, antisense DNA, or a NHE1 RNAi molecule, siRNA, microRNA or other such molecule, or a NHE1 altering ribozyme, or a NHE1 altering DNAzyme.

11. The substance as claimed in claim 7, wherein the NHE1 inhibitory agent is a protein degrader, like a PROTAC, SNIPER, HaloPROTAC, HyT, LYTAC, AUTAC, ATTEC, RIBOTEC, monomeric degrader, double-mechanism degrader, SARD, TF-PROTAC, dual-PROTAC, SERD, bispecific aptamer chimera, AbTAC, GlueTAC, AUTOTAC, CMA-based degrader, MADTAC, ATAC, molecular glue, biodegrader, mRNA for the biodegrader protein, mRNA for the biodegrader protein in a lipid nanoparticle formulation, all targeted at NHE1

12. The substance as claimed in claim 7, wherein the NHE1 inhibitory agent is a gene therapy modifying, completing, replacing, or deleting NHE1 genes.

13. The substance as claimed in one or more of claims 1 to 12, wherein the NHE1 inhibitory agent is a member selected from the group consisting of substances containing the structural units benzoyl-guanidine (based on a phenyl ring), pyrazinoyl-guanidine (based on a pyrazine ring), (piperidin-1-yl)pyrimidine, aminophenoxazine, pyrrolidine, and piperidine.

14. The substance as claimed in one or more of claims 1 to 13, wherein the NHE1 inhibitory agent is a member selected from the group consisting of the compounds Rimeporide, Cariporide, Eniporide, Amiloride, EIPA (5-(N-ethyl-N-isopropyl)amiloride), DMA (5-(N,N-dimethyl)amiloride), MIBA (5-(N-methyl-N-isobutyl)amiloride), HMA (5-(N, N-(hexamethylene)amiloride), 2-aminophenoxazine-3-one, 2-amino-4,4α-dihydro-4α,7-dimethyl-3H-phenoxazine-3-one, Zoniporide, 5-(4-Fluoro-3-methylphenyl)-2-methoxy-4-[4-(5-methyl-1H-imidazol-4-yl)piperidin-1-yl]pyrimidine, and 3-[(Cyclopropylcarbonyl)amino]-N-[2-(dimethylamino)ethyl]-4-[4-(4-methyl-1H-imidazol-5-yl)-1-piperidinyl]benzamide.

15. A pharmaceutical composition, characterized in that it comprises at least one NHE1 inhibitory agent as claimed in one or more of claims 1 to 14.

16. The pharmaceutical composition as claimed in claim 15, characterized in that it comprises (i) at least one NHE1 inhibitory agent according to claim 8, or a pharmaceutically acceptable salt, solvate, hydrate, co-crystal, polymorph, ester, ether, enantiomer, prodrug, or metabolite thereof, and at least one pharmaceutically acceptable diluent or excipient, or (ii) at least one compound of the formulas according to claim 13 or claim 14, or a pharmaceutically acceptable salt, solvate, hydrate, co-crystal, polymorph, ester, ether, enantiomer, prodrug, or metabolite thereof, and at least one pharmaceutically acceptable diluent or excipient.

17. Use of a compound with sodium-hydrogen antiporter 1 inhibitory activity (NHE1 inhibitor) for the manufacture of a medicament for the treatment and prevention of chronic fatigue, exhaustion and/or exertional intolerance, preferably said NHE1 inhibitory compound inhibits the transporter activity of NHE1 at an IC₅₀<50 μM, preferrably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.
